(19) United States
(12) Patent Application Publication
(10) Pub. No.: US 2013/0116568 A1
(43) Pub. Date: May 9, 2013

(54) METHOD AND DEVICE FOR GENERATING ULTRASOUNDS IMPLEMENTING CMUTS, AND METHOD AND SYSTEM FOR MEDICAL IMAGING

(71) Applicants: Dominique Certon, Saint Avertin (FR); Nicolas Senegond, Tours (FR); Franck Teston, Tours (FR)

(72) Inventors: Dominique Certon, Saint Avertin (FR); Nicolas Senegond, Tours (FR); Franck Teston, Tours (FR)

(73) Assignee: UNIVERSITE DE TOURS FRANCOIS RABELAIS, Tours (FR)

(21) Appl. No.: 13/811,307

(22) PCT Filed: Jul. 18, 2011
(86) PCT No.: PCT/FR2011/051705

§ 371 Date: Jan. 21, 2013

(30) Foreign Application Priority Data
Jul. 23, 2010 (FR) 1056040

(51) Int. Cl. A61B 8/00; A61B 8/14

(52) U.S. Cl. CPC A61B 8/4494; A61B 8/14; A61B 8/145
USPC 600/447; 600/459; 600/443

(57) ABSTRACT

A method is provided for generating ultrasounds in a given fluid by using at least one micro-machined capacitive transducer having a membrane and exhibiting a predetermined resonant frequency defined by the membrane-fluid pair, the at least one transducer is fed with an excitation signal of lower frequency than the resonant frequency. A device is provided for generating ultrasounds implementing CMUTs, as well as a method and system for medical imaging.

The present invention relates to a method for generating ultrasound using capacitive micromachined ultrasonic transducers (CMUTs). It also relates to a device for generating ultrasound using such a method. It relates finally to a method and a system for medical imaging using CMUTs.

The field of the invention is the field of the generation of ultrasound using CMUTs.

A CMUT transducer is formed from several hundred, or even a few thousand mechanically isolated “micro-membranes” capable of being actuated by electrostatic forces. These are called CMUTs for Capacitive Micromachined Ultrasonic Transducers. Each CMUT is constituted by a rear electrode formed by a semi-conductor material (generally doped polysilicon), a vacuum cavity having a height H_{gap}, a membrane made of microelectronics material overlaid by an electrode, the membrane/electrode unit constituting the “mobile” part of the device. The material used for the membrane is often silicon nitride but is highly dependent on the technology of fabrication of the device itself. Other materials such as doped polysilicon (in the “wafer bonding” method), a metal or a polymer could be used. CMUTs are now commonly used in the field of medical imaging to excite an organ or a tissue of a human or animal subject. The use of the capacitive micromachined ultrasonic transducers in ultrasound medical imaging is based on the same usage protocols as piezoelectric devices. Typically, the CMUT transducer is polarized with direct current voltage and the sending of a pressure wave is carried out by means of wideband excitation which covers the entire pass band of the transducer. The central frequency of these devices, i.e. the resonance frequency, is defined by the membrane/fluid pair which plays the role of a spring/mass system where the elasticity depends only on the properties of the membrane and the mass of the fluid. This mass effect is moreover dependent on the effects of mutual interactions between membranes the consequence of which is to create cut-off frequencies in the pass band of the transducer.

However, the generation of low-frequency ultrasound, for example ultrasound at frequencies less than or equal to 2 MHz requires membranes having a low mechanical rigidity that can be obtained either by increasing their width, or by reducing their thickness or using materials that have a low Young's modulus. The low resonance frequency devices generally have a low functional capability. In fact, as their mechanical rigidity is relatively low, the membranes are subjected to the pressure of the outside air and are thus deformed by several tens of nanometres or even around a hundred. The deformation can lead to the membrane becoming jammed at the base of the cavity, thus rendering the device unusable. In order to compensate for this deflection, the height of the cavity can be increased in order to retain a “free” space between the membrane and the rear of the cavity, but this leads to a significant increase in the supply voltages necessary to drive the CMUTs. The increase in the supply voltage reduces the possibilities for use, as a very high voltage of use (several hundred volts) requires specific voltage supply means. In order to avoid this deflection, a gas, the pressure of which is equal to average outside pressure, can be maintained in the cavity. However, the dynamic damping effects linked to the presence of this gas significantly change the resonance of the device and require an architecture of complex CMUTs intended to eliminate these effects (perforation of the rear cavity). These solutions are easy to implement for the very low-frequency devices (less than 100 kHz) but relatively costly and difficult to carry out for higher frequencies.

A purpose of the present invention is to remedy the above drawbacks. A another purpose of the present invention is to propose a method and a device for generating ultrasound with at least one CMUT transducer that is easier to fabricate, cheaper and operates with a supply voltage that is more accessible and acceptable for low-voltage supplies, while making it possible to obtain satisfactory useful pressure levels.

The invention proposes to achieve the above-mentioned purposes by a method for generating ultrasound in a given fluid using at least one capacitive micromachined ultrasonic transducer (CMUT) comprising a membrane and having a predetermined resonance frequency defined by the membrane-fluid pair, characterized in that said at least one transducer is supplied with an excitation signal having a frequency lower than said central frequency.

Of course the frequency f of the ultrasound wave generated is lower than the resonance frequency f₀ and more particularly equal to the frequency of the excitation signal.

The invention relates to the transducers the membranes of which have the same architecture such that they all have the same and a single resonance frequency.

According to the invention the CMUT transducer comprises at least one capacitive micro-machined (CMUT) cell, also called “micro-membrane”, that is mechanically isolated and capable of actuation by electrostatic forces.

The inventors of the present invention surprisingly found, on the basis of experimental results obtained in air and in water, that a capacitive micromachined ultrasonic transducer is capable of producing high-amplitude displacements, well below its membrane-fluid interaction frequency. Unlike the piezoelectric systems which have a high mechanical stiffness, it is not necessary for the membrane of the CMUT transducer to resonate in order to produce displacements that are sufficiently large to generate pressure at significant levels.

Thus, the inventors propose an ultrasound generation based on the exploitation of the purely “elastic” behaviour mode of the membranes of the CMUT transducers, which are capable of producing the entire gap height as the amplitude of displacements. Moreover, the inventors also found that in the low-frequency range, each membrane behaves as an “ideal” pressure point source, which means that a single parameter sets the amplitude of the ultrasound pressure emitted: the number of CMUT membranes present in an array. In other words: for an equivalent surface area, this is the coverage rate and the average amplitude of the displacements which define the radiated ultrasound intensity.

Thus, when generating ultrasound from one or more CMUT transducers excited with an excitation signal below the central frequency of the transducer(s), it is not necessary to design acoustic transducers as complex, costly and difficult to use or to implement as if they were used at their resonance frequency. The invention therefore makes it possible to generate ultrasound in a simpler and less costly manner.

More particularly, the inventors found that the frequency of the excitation signal is advantageously at least 20% or even 50% lower than the central frequency of the at least one capacitive micromachined ultrasonic transducer.

Even more particularly the inventors found that the frequency f of the excitation signal f₀ can be lower than one half of the resonance frequency, and more particularly 0.2 f₀≦f<0.5 f₀, and more particularly 0.3 f₀≦f<0.5 f₀, 0.4 f₀≦f<0.5 f₀.

The inventors have succeeded in generating ultrasound, with a CMUT transducer having a single resonance frequency f₀, at frequencies well below f₀, typically below f₀/2. The property exploited for this method of generation, called “forced elastic regime”, is the ability of CMUT technologies to produce local displacements of several tens, or even around a hundred nanometres without requiring the membranes to resonate. This procedure then allows the generation of low-frequency ultrasound waves in a wide frequency band, independently of the geometry and topology of the diaphragm.

For example, with respect to a transducer the resonance frequency of which is 4 MHz, it is equally possible with this same device to emit an ultrasound wave at 1 MHz, or at 1.5 MHz without necessarily needing to design a device having several resonance frequencies.

In order to illustrate the pressure levels transmitted in water, the following parameters of the transducer are considered:
circular transducer of radius 10 mm,
membrane the resonance frequency of which is 4 MHz in water,
peak-to-peak displacement of the membranes of 180 nm, i.e. an average displacement of 60 nm,
the membrane fill factor on the transducer is 50%.

At 1 MHz the pressure transmitted at the focal point is 1 MPa and at 1.5 MHz it is 1.5 MPa.

Thus, in a particular embodiment, with a CMUT transducer having a central frequency of 4 MHz in water and 12 MHz in air, the inventors have carried out ultrasound generation at frequencies comprised between:
200 kHz and 2 MHz in water, and
200 kHz and 1 MHz in air,
   with satisfactory useful pressure levels. In fact, the useful pressure levels obtained for a radiating surface area equivalent to 100 mm² at an excitation frequency of 500 kHz are greater than or equal to:
220 dB (reference pressure, P_{ref}=1 μPa) in an aqueous medium at a distance of 10 cm, and
70 dB (P_{ref}=20 μPa) in air at a distance of 30 cm.

Advantageously, the at least one capacitive micromachined ultrasonic transducer can be designed so that its central frequency is greater than or equal to 4 MHz and with a gap height comprised between 100 nm and 300 nm, said at least one transducer being excited with an excitation signal having a frequency less than 2 MHz in order to generate ultrasound having frequencies comprised between 200 kHz and 2 MHz.

Moreover, according to the invention, the supply voltage of the at least one capacitive micromachined ultrasonic transducer can be comprised between 1 V and 150 V. These voltages are lower voltages than those used in the state of the art to supply CMUT transducers for generating low-frequency ultrasound, in particular for frequencies less than 2 MHz in water and 1 MHz in air.

The method according to the invention can be used for generating ultrasound having frequencies less than 1 MHz in a gaseous medium with an excitation signal comprised between 200 kHz and 1 MHz.

In this case the supply voltage can be comprised between 50 and 150 V with a gap height H_{gap} comprised between 100 and 300 nm.

The method according to the invention can also be used for generating ultrasound having frequencies less than 2 MHz in a liquid or aqueous medium with an excitation signal comprised between 200 kHz and 2 MHz.

In this case, the supply voltage can be comprised between
50 and 150 V for a gap height H_{gap} comprised between approximately 100 nm and approximately 200 nm.
100 and 150 V for a gap height H_{gap} comprised between approximately 200 nm and approximately 300 nm.

According to a particular implementation, the method according to the invention allows the generation of ultrasound:
having a useful pressure level, for a radiating surface area equivalent to 100 mm² at an excitation frequency of 500 kHz, greater than or equal to:
   70 dB in air at a distance of 30 cm, and
   220 dB in an aqueous medium at a distance of 10 cm;
having a frequency:
   less than or equal to 1 MHz in a gaseous medium, and
   less than or equal to 2 MHz in an aqueous medium; using at least one capacitive micromachined ultrasonic transducer (CMUT) designed so that it has:
a resonance frequency or central frequency greater than or equal to 4 MHz, and
a gap height comprised between 100 nm and 300 nm,
   said method comprising supplying said capacitive micromachined ultrasonic transducer with a supply voltage comprised between 1V and 150 V having a frequency comprised between 200 kHz and 1 MHz in the gaseous medium and 200 kHz and 1 MHz in the aqueous medium.

According to another aspect of the invention, a method is proposed for the medical imaging of a tissue or an organ of a human or animal subject comprising the following steps:
generating ultrasound in accordance with the method according to the invention in order to excite said tissue or organ, and
taking at least one image of said organ or tissue with imaging means when said organ or tissue is thus excited.

According to another aspect of the invention, a device is proposed for generating ultrasound in a given fluid using at least one capacitive micromachined ultrasonic transducer (CMUT) comprising a membrane and having a predetermined resonance frequency defined by the membrane-fluid pair, characterized in that said transducer is supplied with an excitation signal having a frequency less than said central frequency, preferably at least 20% or even 50%.

Advantageously, the device according to the invention can comprise at least one capacitive micromachined ultrasonic transducer (CMUT) designed so that it has:
a resonance frequency or central frequency greater than or equal to 4 MHz in water, and
a gap height comprised between 100 nm and 300 nm.

According to the invention, the transducer is supplied with a supply voltage comprised between 1V and 150 V delivered by supply means.

According to a particular example of the device according to the invention, when the device according to the invention is used for generating ultrasound in an aqueous or liquid medium, the capacitive micromachined ultrasonic transducer has:
a gap height of 100 nm,
an excitation voltage of 50 V,
a membrane width comprised between 13 and 35 μm,
a membrane thickness comprised between 200 and 800 nm, and
A Young's modulus of 200 GPa.

According to another particular embodiment of the device according to the invention, when the device according to the invention is used for generating ultrasound in an aqueous or liquid medium, the capacitive micromachined ultrasonic transducer has:
a gap height of 200 nm,
an excitation voltage of 100 V,
a membrane width comprised between 13 and 35 μm,
a membrane thickness comprised between 200 and 800 nm, and
A Young's modulus of 200 GPa.

According to yet another embodiment, when the device according to the invention is used for generating ultrasound in an aqueous or liquid medium, the capacitive micromachined ultrasonic transducer having:
a gap height of 300 nm,
an excitation voltage of 100 V,
a membrane width comprised between 20 and 30 μm,
a membrane thickness comprised between 300 and 550 nm, and
A Young's modulus of 200 GPa.

According to another particular embodiment of the device according to the invention, when the device according to the invention is used for generating ultrasound in a gaseous medium, the capacitive micromachined ultrasonic transducer has:
a gap height of 100 nm,
an excitation voltage of 50 V,
a membrane width comprised between 10 and 35 μm,
a membrane thickness comprised between 200 and 800 nm, and
a Young's modulus of 200 GPa.

According to yet another particular embodiment of the device according to the invention, when the device according to the invention is used for generating ultrasound in a gaseous medium, the capacitive micromachined ultrasonic transducer has:
a gap height of 200 nm,
an excitation voltage of 50 V,
a membrane width comprised between 20 and 40 μm,
a membrane thickness comprised between 300 and 600 nm, and
a Young's modulus of 200 GPa.

According to yet another particular embodiment of the device according to the invention, when the device according to the invention is used for generating ultrasound in a gaseous medium, the capacitive micromachined ultrasonic transducer has:
a gap height of 300 nm,
an excitation voltage of 100 V,
a membrane width comprised between 20 and 30 μm,
a membrane thickness comprised between 300 and 600 nm, and
a Young's modulus of 200 GPa.

According to a particularly advantageous embodiment, the device according to the invention can comprise:
a first supply module provided to supply the capacitive micromachined ultrasonic transducer with an excitation signal having a frequency less than said central frequency,
a second supply module provided to supply the capacitive micromachined ultrasonic transducer with an excitation signal having a frequency centred around said central frequency, and
selection means for selecting one of said supply modules such that said capacitive micromachined ultrasonic transducer is supplied by only one of said supply modules at a time.

According to another particularly advantageous embodiment, the device according to the invention can comprise:
at least one first and at least one second capacitive micromachined ultrasonic transducer having an identical central frequency,
a first supply module provided to supply said at least one first capacitive micromachined ultrasonic transducer with an excitation signal having a frequency less than said central frequency,
a second supply module provided to supply said at least one capacitive micromachined ultrasonic transducer with an excitation signal having a frequency centred around said central frequency.

According to yet another aspect of the invention an ultrasound medical imaging system is proposed comprising:
at least one device for generating ultrasound according to the invention in order to excite a tissue or an organ of a human or animal subject, and
imaging means for taking images of said tissue or organ when said organ is excited. The imaging means can comprise MRI imaging means or any other imaging means used in the field of ultrasound medical imaging.

Other advantages and characteristics will become apparent on examination of the detailed description of an embodiment which is in no way imitative, and the attached diagrams, in which:

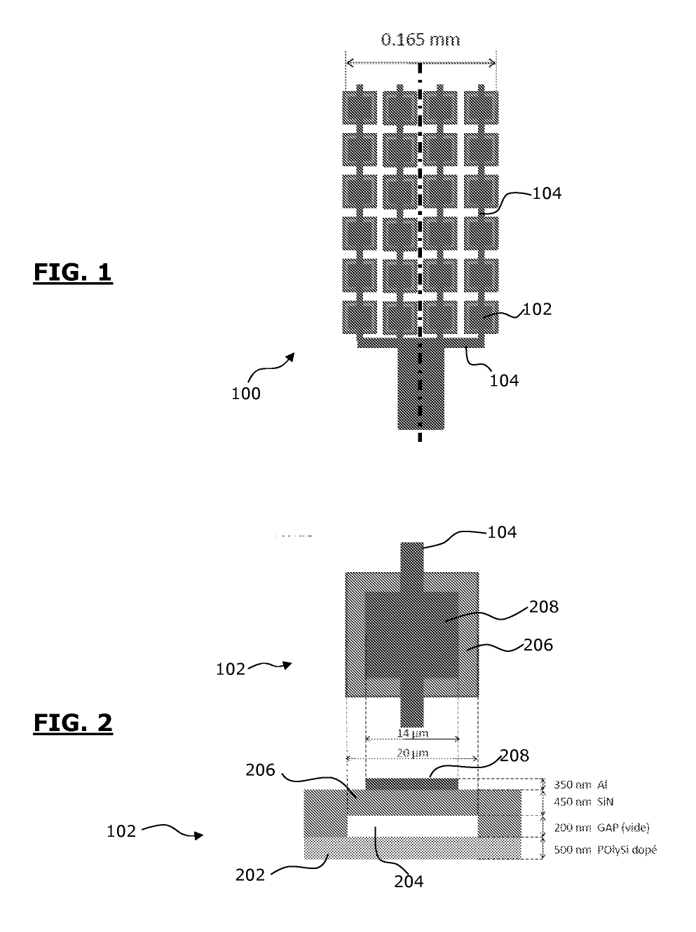

FIG. 1 is a diagrammatic representation of an example capacitive micromachined ultrasonic transducer comprising a plurality of elementary CMUT cells ; and

FIG. 2 is a diagrammatic representation of an elementary CMUT cell in top view and in cross-sectional view;

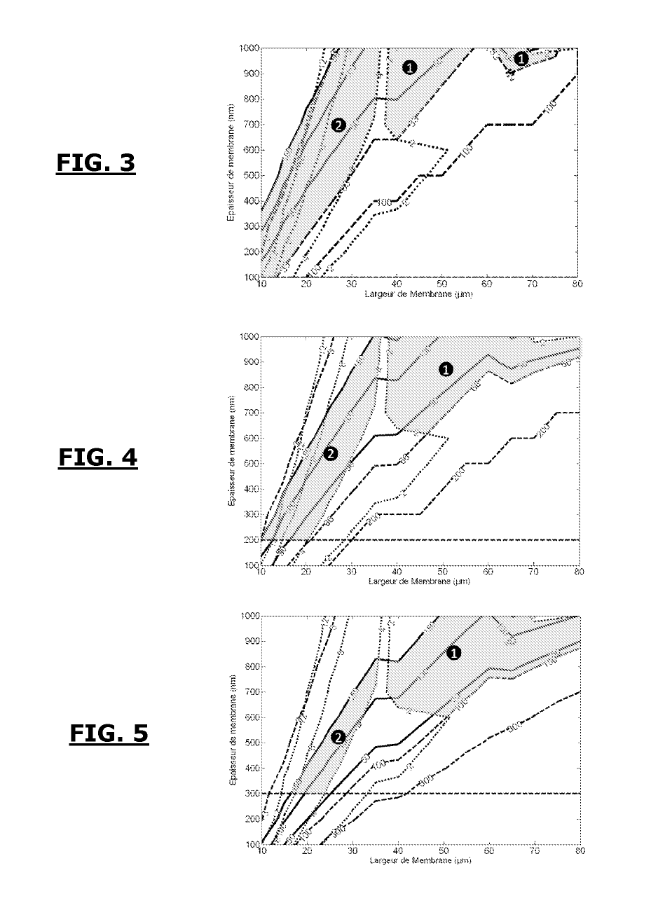

FIGS. 3 to 5 are graphs representing simulation results in water of a CMUT transducer for different gap heights (or cavity heights) as a function of the membrane width, membrane height, supply voltage and central frequency of the CMUT transducer, for a constant Young's modulus;

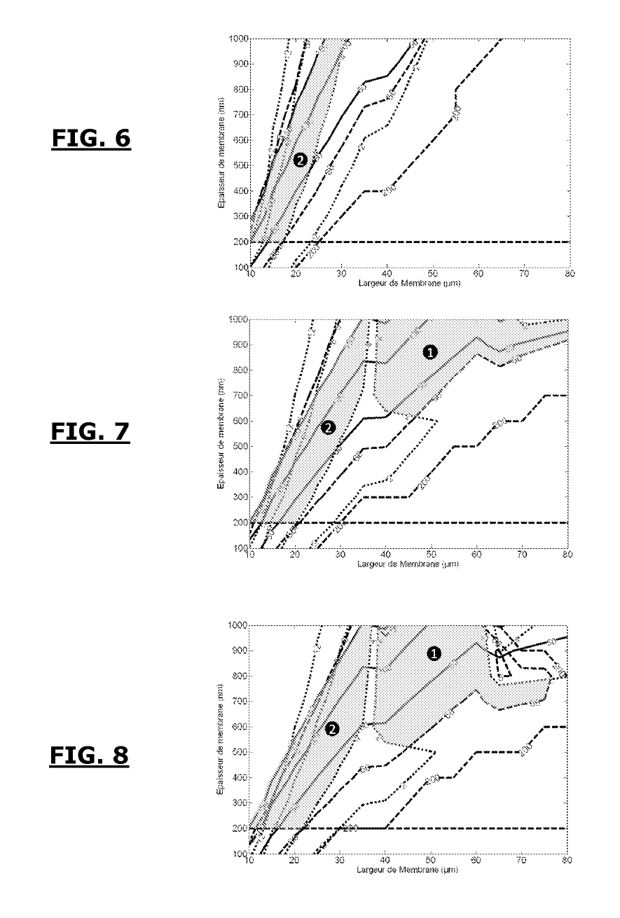

FIGS. 6 to 8 are graphs representing simulation results in water of a CMUT transducer for different Young's moduli as a function of the membrane width, membrane height, supply voltage and central frequency of the CMUT transducer, for a constant gap height (or cavity height);

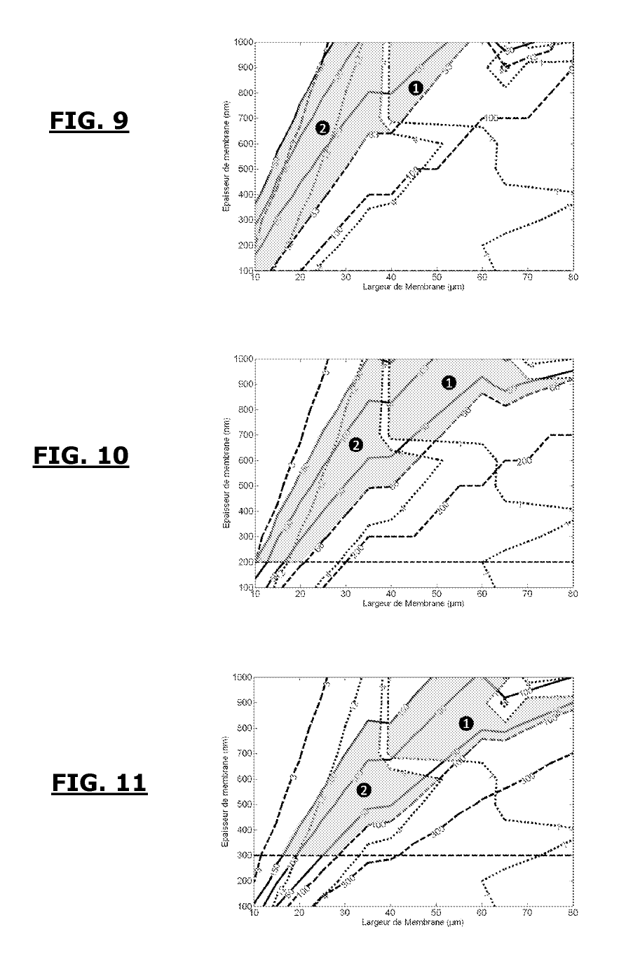

FIGS. 9 to 11 are graphs representing simulation results in air of a CMUT transducer for different gap heights (or cavity heights) as a function of the membrane width, membrane height, supply voltage and central frequency of the CMUT transducer, for a constant Young's modulus;

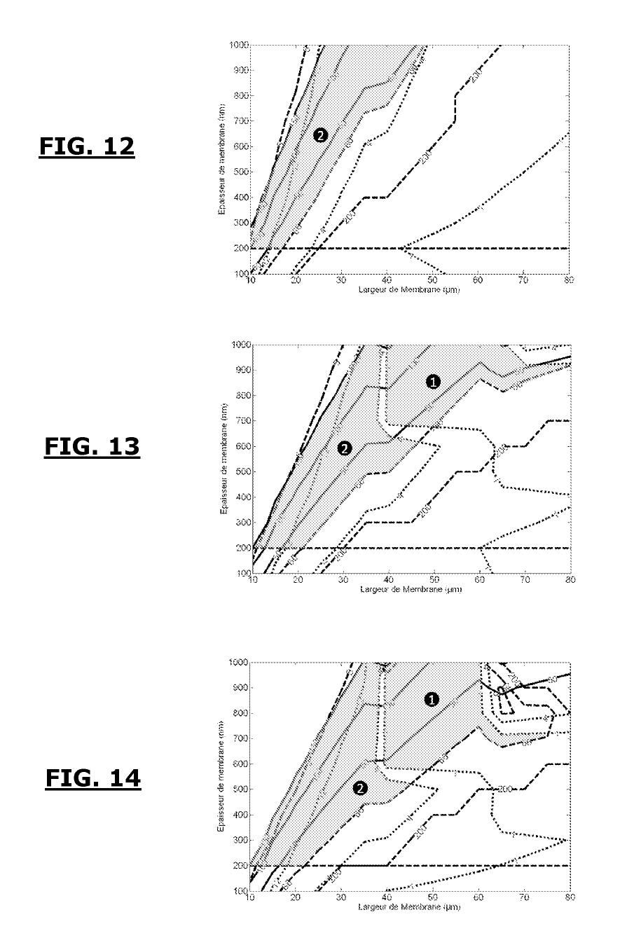

FIGS. 12 to 14 are graphs representing simulation results in air of a CMUT transducer for different Young's moduli as a function of the membrane width, membrane height, supply voltage and central frequency of the CMUT transducer, for a constant gap height (or cavity height);

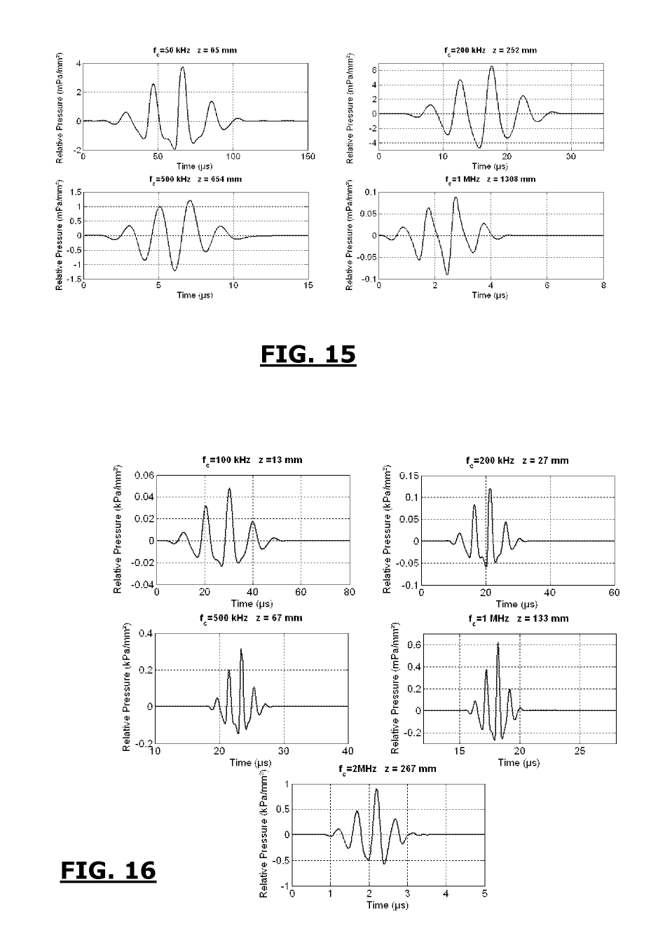

FIG. 15 is a group of graphs representing values of the pressure field radiated in a gaseous medium by an excited CMUT transducer, according to the invention, in the forced elastic regime;

FIG. 16 is a group of graphs representing values of the pressure field radiated in a liquid medium by an excited CMUT transducer, according to the invention, in the forced elastic regime,

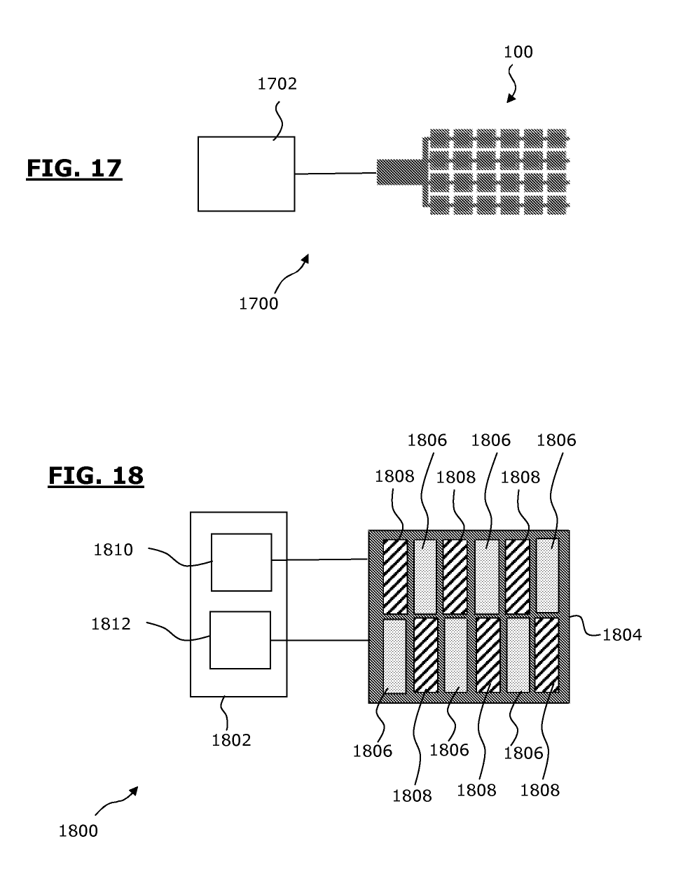

FIG. 17 is a diagrammatic representation of an example device according to the invention; and

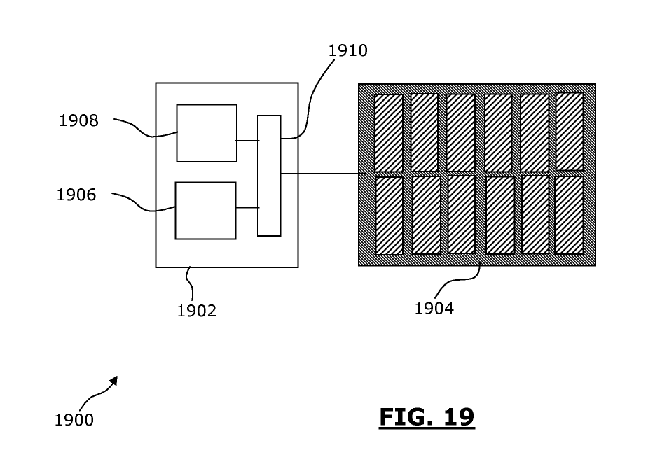

FIGS. 18 and 19 are representations of two embodiments of a double-function device according to the invention.

A CMUT transducer is formed by several hundred, even a few thousand mechanically isolated “micro-membranes” capable of being actuated by electrostatic forces. These are called CMUTs, for Capacitive Micromachined Ultrasonic Transducers. These membranes are simple capacitive microphones, the operating principle of which is similar to that of the devices used in audio for applications in air. There are however appreciable differences, as the cavities on which the membranes rest are at zero pressure and are isolated from the outside, thus also allowing use in a fluid medium.

FIG. 1 is a diagrammatic representation of an example of a capacitive micromachined ultrasonic transducer 100.

The CMUT transducer 100 comprises, non-limitatively, 24 elementary cells 102, or micro-membranes, having a square geometry arranged in 6 rows of 4. The width of the transducer 100 is 0.165 mm.

The CMUT transducer also comprises supply lines 104 of each of the cells.

FIG. 2 is a diagrammatic representation of an elementary CMUT cell 102 in a top view and cross sectional view;

The elementary cell 102 comprises:
a rear electrode 202 formed by a semi-conductor material, for example doped polysilicon, having a thickness of 500 nm for example;
a vacuum cavity 204 having a given height called gap height H_{gap}, having a value of 200 nm for example;
a membrane 206 made of microelectronic material, for example having a thickness of 450 nm; and
a front electrode 208 also called a “mobile” electrode having a thickness of 350 nm for example.

The material used for the membrane is for example silicon nitride but is highly dependent on the technique of fabrication of the device. Other materials such as doped polysilicon (in wafer bonding), a metal or a polymer could be used.

The mobile electrode 208 can be made of aluminium, or any other type of conductor material that is compatible with the use. Similarly, the materials used for producing the mobile electrode 208 are distinguished only by their Young's modulus.

Finally, it should be noted that the metallization on the front face on each membrane can be from 100% of the surface area to a few percent. It is often accepted that 50% metallized surface is a good compromise between stiffness/mass and effectiveness of the electrostatic forces. It is important to specify that, from a mechanical point of view, changing the thickness of the membranes or the Young's modulus of the materials or the metallization rate is defined by an overall parameter called flexural rigidity, which is the single useful mechanical parameter of these microsystems.

The two design parameters of these microsystems are:
the resonance frequency in air or in water according to usage, and
the collapse voltage Vc which constitutes the maximum excitation voltage of the CMUTs, beyond which the membranes cannot remain in equilibrium between electrostatic forces and mechanical forces and touch the “base” of the cavity.

The resonance frequency depends:
on the geometry,
on the surface area,
on the flexural rigidity of the membranes,
on the mass of the membranes (in air) and on that of the fluid (in water).

The collapse voltage depends:
on the geometry,
on the surface area,
on the flexural rigidity of the membranes,

The collapse voltage Vc increases if the flexural rigidity increases and/or if the surface area increases.

The present invention proposes, in the present example, compromises or compromise areas of interest, constituting “technical pathways” of interest for low-frequency work where the membrane of each of the CMUT cells is used in forced regime and not in “resonant” mode. In air, this corresponds to the capacity for generating significant amplitude displacements for frequencies less than 1 MHz while the resonance frequency is considerably greater. In water, the low frequency is situated below 2 MHz. This then corresponds to the ability to generate significant low-frequency displacements while the resonance is situated well above 2 MHz, typically above 4 MHz.

Thus, the invention proposes to produce transducers capable of generating low-frequency ultrasound in air and in water, relying on lower-cost production methods, less complex than the devices of the state of the art, in this case the techniques of surface micro-machining over very great widths or using particularly flexible materials.

In fact, the use of the “resonant” mode as low-frequency source in the state of the art imposes production methods that are much more costly, such as the “wafer bonding” type techniques. These methods offer compromises in terms of width (of the order of a millimetre) and membrane thickness (typically 50 μm) of interest for achieving a resonance frequency which is low, with however very high supply voltages (greater than 500 Volts).

Simulations carried out by the inventors make it possible to show and identify technology pathways allowing the generation of low-frequency ultrasound, i.e. less than 1 MHz in air and 2 MHz in water, using CMUT ultrasound transducers the central frequencies of which are well above the generated ultrasound frequencies.

These simulations make it possible to identify, as a function of the gap height H_{gap}, the Young's modulus, the membrane width, the membrane thickness and the central frequency of the CMUT transducers, the compromises obtained for a supply voltage less than or equal to 150 V while obtaining a useful pressure level for a radiation surface area equivalent to 100 mm² at an excitation frequency of 500 kHz that is greater than or equal to:
70 dB in air at a distance of 30 cm, and
220 dB in water at a distance of 10 cm.

Thus, FIGS. 3 to 5 are graphs representing simulation results in water of a CMUT transducer for different gap heights (or cavity height) as a function of the membrane width, membrane height, supply voltage and central frequency of the CMUT transducer, for a constant Young's modulus of 200 GPa;

FIGS. 3 to 5 show the simulation results respectively for gap heights of H_{gap}=100 nm, 200 nm and 300 nm.

In FIGS. 3 to 5:
the solid lines correspond to the curves of the collapse voltage value levels in Volts,
the close-dotted lines correspond to the curves of the resonance frequency levels in MHz,
the wide-dotted lines correspond to the curves of the initial deflection levels in nm.

In each of these figures, the grey area marked (2) corresponds to the technical compromise values for generating ultrasound having a frequency less than or equal to 2 MHz with transducers having a central frequency greater than or equal to 4 MHz.

With respect to a gap height of H_{gap}=100 nm, the area marked (2) is bounded by the coordinate points [membrane width, membrane thickness]: [10 μm, 100 nm], [10 μm, 400 nm], [30 μm, 600 nm], [30 μm, 1000 nm].

With respect to a gap height of H_{gap}=200 nm, the area marked (2) is bounded by the coordinate points [membrane width, membrane thickness]: [10 μm, 200 nm], [15 μm, 200 nm], [25 μm, 400 nm], [35 μm, 1000 nm].

With respect to a gap height of H_{gap}=300 nm, the area marked (2) is bounded by the coordinate points [membrane width, membrane thickness]: [15 μm, 300 nm], [25 μm, 300 nm], [30 μm, 600 nm], [30 μm, 800 nm].

FIGS. 9 to 11 are graphs representing simulation results obtained in air under the same conditions as for FIGS. 3 to 5.

In FIGS. 9 to 11:
the solid lines correspond to the curves of the collapse voltage value levels in Volts,
the close-dotted lines correspond to the curves of the resonance frequency levels in MHz,
the wide-dotted lines correspond to the curves of the initial deflection levels in nm.

In each of these figures, the grey area marked (2) corresponds to the technical compromise values for generating ultrasound having a frequency less than or equal to 1 MHz with transducers having a central frequency greater than or equal to 4 MHz.

With respect to a gap height of Hgap=100 nm, the area marked (2) is bounded by the coordinate points [membrane width, membrane thickness]: [10 μm, 100 nm], [15 μm, 100 nm], [35 μm, 700 nm], [25 μm, 1000 nm].

With respect to a gap height of Hgap=200 nm, the area marked (2) is bounded by the coordinate points [membrane width, membrane thickness]: [10 μm, 200 nm], [15 μm, 200 nm], [40 μm, 600 nm], [35 μm, 1000 nm].

With respect to a gap height of Hgap=300 nm, the area marked (2) is bounded by the coordinate points [membrane width, membrane thickness]: [15 μm, 300 nm], [25 μm, 300 nm], [45 μm, 600 nm], [40 μm, 700 nm].

FIGS. 6 to 8 are graphs representing results of simulation in water of a CMUT transducer for different Young's moduli as a function of the membrane width, membrane height, supply voltage and central frequency of the CMUT transducer, for a constant gap height (or cavity height) of 200 nm.

FIGS. 6 to 8 show the simulation results respectively for Young's modulus values of E_{mb}=50 GPa, 200 GPa and 300 GPa.

In FIGS. 6 to 8:
the solid lines correspond to the curves of the collapse voltage value levels in Volts,
the close-dotted lines correspond to the curves of the resonance frequency levels in MHz,
the wide-dotted lines correspond to the curves of the initial deflection levels in nm.

In each of these figures, the grey area marked (2) corresponds to the technical compromise values for generating ultrasound having a frequency less than or equal to 2 MHz with transducers having a central frequency greater than or equal to 4 MHz.

With respect to a Young's modulus E_{mb}=50 GPa, the area marked (2) is bounded by the coordinate points: [membrane width, membrane thickness]: [10 μm, 200 nm], [15 μm, 200 nm], [30 μm, 1000 nm], [25 μm, 1000 nm].

With respect to a Young's modulus E_{mb}=200 GPa, the area marked (2) is bounded by the coordinate points [membrane width, membrane thickness]: [10 μm, 200 nm], [15 μm, 200 nm], [25 μm, 400 nm], [35 μm, 1000 nm].

With respect to a Young's modulus Emb=300 GPa, the area marked (2) is bounded by the coordinate points [membrane width, membrane thickness]: [10 μm, 200 nm], [20 μm, 200 nm], [35 μm, 600 nm], [35 μm, 1000 nm].

FIGS. 12 to 14 are graphs representing simulation results obtained in air, under the same conditions as for FIGS. 6 to 8.

In FIGS. 12 to 14:
the solid lines correspond to the curves of the collapse voltage value levels in Volts,
the close-dotted lines correspond to the curves of the resonance frequency levels in MHz,
the wide-dotted lines correspond to the curves of the initial deflection levels in nm.

In each of these figures, the grey area marked (2) corresponds to the technical compromise values for generating ultrasound having a frequency less than or equal to 1 MHz with transducers having a central frequency greater than or equal to 4 MHz.

With respect to a Young's modulus E_{mb}=50 GPa, the area marked (2) is bounded by the coordinate points [membrane width, membrane thickness]: [10 μm, 200 nm], [15 μm, 200 nm], [40 μm, 1000 nm], [25 μm, 1000 nm].

With respect to a Young's modulus E_{mb}=200 GPa, the area marked (2) is bounded by the coordinate points [membrane width, membrane thickness]: [10 μm, 200 nm], [15 μm, 200 nm], [40 μm, 600 nm], [35 μm, 1000 nm].

With respect to a Young's modulus E_{mb}=300 GPa, the area marked (2) is bounded by the coordinate points: [membrane width, membrane thickness]: [10 μm, 200 nm], [20 μm, 200 nm], [35 μm, 500 nm], [30 μm, 1000 nm].

FIG. 15 is a group of graphs representing values of the pressure field radiated in air by an excited CMUT transducer according to the invention in the forced elastic regime. To this end, a transducer having a square geometry of size 30×30 mm² comprising a 2D network of square membranes 20×20 μm² with a periodicity of 30 μm, i.e. a coverage rate of 45% and therefore an average active surface area of 405 mm² was used. For the 4 measured frequencies, namely 50 kHz, 200 kHz, 500 kHz and 1 MHz, the pressure field was measured at the near-field limit, along the axis of the transducer, i.e. respectively z=65, 252, 654 and 1308 mm for the respective frequencies of 50 kHz, 200 kHz, 500 kHz and 1 MHz.

FIG. 15 shows that the emitted pressure field accurately follows the excitation frequency initially applied to the CMUT transducer. The pressure values reached are comparable to the values required for operation of these devices in air. By way of reference, the standards for transmission in air specify that a reference value for the SPL (Sound Pressure Level) is 20 μPa at a distance of 30 cm and that a data transmission application requires a pressure of the order of 100-120 dB i.e. between 2 and 20 Pa.

FIG. 16 is a group of graphs representing values of the pressure field radiated in water by an excited CMUT transducer, according to the invention, in the forced elastic regime. The measurements were carried out with a transducer having a square geometry with a surface area of 20×20 mm², with a coverage rate of 45%. The pressure field was determined at the near-field limit at z=13, 27, 67, 133 and 267 mm for the respective frequencies of 100, 200, 500, 1 and 2 MHz.

The pressure field emitted accurately follows the excitation frequency initially applied to the CMUT transducer. The pressure values reached are comparable to the values required for operation of these devices in water.

The invention makes it possible to replace the conventional piezo-electric materials with silicon components on which are etched thousands of capacitive microcomponents capable of vibrating. This CMUT (Capacitive Micromachined Ultrasonic Transducers) technology has a remarkable property for these applications: at a low frequency, the CMUT membranes, more elastic than inertial, are capable of deformation over amplitudes of a few hundred nanometres for excitation voltages of less than 100 Volts.

Advantageously, the invention can be used to produce low-frequency sensors (100 kHz-2 MHz) based on CMUT technologies.

CMUTs are used under operating conditions that are different from those used in medical imaging where the emission is a wide band excitation (greater than 20 MHz), the amplitude of which is typically 150 Volt. The invention makes it possible to use them under quasi-static conditions (low band excitation <2 MHz) so as to impose high-amplitude displacements on the membranes, close to the cavity height. These technologies offer several advantages which make them particularly advantageous for low-frequency applications:
The space requirement of the transducer is linked only to the thicknesses of the wafer on which the CMUTs are etched, and to the connecting elements.
The risks of overheating of the transducer are much lower than those of ceramic technology sensors.
By design, the CMUT arrays have almost non-existent inter-element acoustic couplings.
It is then possible to connect two different and complementary functions onto the same device, one dedicated to low frequency (therapy) and the other to high frequency (imaging/diagnostics).

FIG. 17 is a representation of an example device 1700 for the excitation of a tissue and/or an organ of a human or animal subject implementing the invention.

The device 1700 comprises an acoustic transducer 100 as shown in FIG. 1 and means 1702 for supplying the transducer 100 with an excitation signal having a frequency less than the central frequency of the transducer 100.

As specified above, the invention also makes it possible to connect onto the same excitation device two different and complementary functions, namely:
a first function dedicated to low frequency, for example 1 MHz, for the purpose of providing a therapy, and
a second function dedicated to high frequency, for example comprised between 4 and 8 MHz, for carrying out imaging or diagnostics.

FIG. 18 is a diagrammatic representation of a first example device allowing the two above-mentioned functions to be carried out. The device 1800, shown in FIG. 18, comprises supply means 1802 and a set of acoustic transducers 1804. Each of the acoustic transducers 1804 comprises CMUT membranes having exactly the same topology as the other acoustic transducers 1804, and therefore the same central frequency, for example comprised between 4 and 8 MHz.

In order to carry out the two functions mentioned above, a part 1806 of the acoustic transducers 1804 is used for generating a low-frequency ultrasonic beam, for example of 1 MHz, used in therapy. These transducers 1804 are therefore used in elastic mode, below their central frequency.

The other part 1808 of the acoustic transducers 1804 is used for generating a high-frequency ultrasonic beam, for example of 4 to 8 MHz, used in ultrasound imaging. The acoustic transducers 1808 are therefore excited at their central frequency or around this central frequency.

As the two functions using CMUT membranes have exactly the same topology, the design and fabrication of the double-function device are simplified as all the cells are exactly identical. Such a device has the advantage of being able to separate the low-frequency emission electronics for therapy from the electronics dedicated to conventional ultrasound imaging.

In fact, for the therapy part, the low-frequency signals make it possible to scan the entire height of the cavity in order to benefit from an adequate ultrasound pressure level. Consequently, in the elastic regime, a polarization voltage equal to the collapse voltage divided by two (Vc/2) and a dynamic amplitude corresponding to 100% of Vc is used. The acoustic transducers 1806 are therefore used in the elastic regime and are excited with an excitation signal having a frequency below their central frequency, supplied by a supply module 1810.

For the imaging part, the acoustic transducers 1808 are excited by an excitation signal of the wide band impulse type, centred on the central frequency of the CMUTs combined with a polarization voltage corresponding to 80% Vc and supplied by a supply module 1812 to the acoustic transducers 1808. This choice promotes reception sensitivity. The amplitudes of excitation used for the imaging transducers 1808 are lower than the amplitudes used for the therapy transducers 1806 as the transducers 1808 are used in “resonant” mode and as the pressure is proportional to the square of the frequency, it is higher on that basis.

FIG. 19 is a diagrammatic representation of a second example device allowing the two above-mentioned functions to be performed. The device 1900 makes it possible to perform the two functions by separating the two functions in time.

To this end, the device 1900 comprises supply means 1902 and a set of identical ultrasound transducers 1904. Each ultrasound transducer 1904 is used both in therapy and in imaging/diagnostics and has the same central frequency.

The supply means 1902 comprise a first supply module 1906 supplying a low-frequency signal for therapy, for example 1 MHz, and a second supply module 1908 supplying a high-frequency signal for imaging/diagnostics, for example comprised between 4 MHz and 8 MHz. The supply means 1902 also comprise a selection module 1910 making it possible to select the source of supply of the transducers 1904 manually or automatically and optionally programmable.

Thus, when the device 1900 is used in therapy, the selection module 1910 chooses the supply module 1906. In the event that the device 1900 is used in imaging/diagnostics the selection module 1910 chooses the supply module 1908.

The advantage of the device 1900 is linked to the orientation of the high- and low-frequency beams, which with the device 1900 are accurately superimposed.

Of course the invention is not limited to the non-limitative embodiments described above.

1. A method for generating ultrasound in a given fluid, comprising: using at least one capacitive micromachined ultrasonic transducer having a membrane and having a predetermined resonance frequency defined by the membrane-fluid pair, at least one transducer is supplied with an excitation signal having a frequency lower than said resonance frequency so as to generate an ultrasound wave having a frequency lower than said resonance frequency.

2. The method according to claim 1, characterized in that the frequency of the excitation signal is at least 20 to 50% lower than the resonance frequency of the at least one capacitive micromachined ultrasonic transducer.

3. The method according to claim 1, characterized in that the at least one capacitive micromachined ultrasonic transducer is designed such that its resonance frequency is greater than or equal to 4 MHz and has a gap height comprised between 100 nm and 300 nm, said at least one transducer being excited with an excitation signal having a frequency less than 2 MHz.

4. The method according to claim 1, characterized in that a supply voltage of the at least one capacitive micromachined ultrasonic transducer is comprised between 1V and 150 V.

5. Use of the method according to claim 1, for generating ultrasound having frequencies less than 1 MHz in a gaseous medium with an excitation signal comprised between 200 kHz and 1 MHz.

6. The use according to claim 5, characterized in that the supply voltage is comprised between 50 and 150 V.

7. Use of the method according to claim 1, for generating ultrasound having frequencies less than 2 MHz in a liquid medium with an excitation signal comprised between 200 kHz and 2 MHz.

8. The use according to claim 7, characterized in that the supply voltage is comprised between:
50 and 150 V for a gap height around 100 nm; and
100 and 150 V for a gap height around 200 nm.

9. A method for medical imaging of a tissue or an organ of a human or animal subject comprising the following steps:
generating ultrasound according to any one of the previous claims for exciting said tissue or organ; and
taking at least one image of said organ or tissue with imaging means when said organ or tissue is excited.

10. A device for generating ultrasound in a given fluid, comprising: at least one capacitive micromachined ultrasonic transducer including a membrane and having a predetermined resonance frequency defined by the membrane-fluid pair, and having moreover a suitable supply for supplying said transducer with an excitation signal having a frequency lower than said resonance frequency so as to generate an ultrasound wave having a frequency lower than said resonance frequency.

11. The device according to claim 10, characterized in that it comprises at least one capacitive micromachined ultrasonic transducer designed so that it has:
a resonance frequency or central frequency greater than or equal to 4 MHz; and
a gap height comprised between 100 nm and 300 nm;
said transducer being supplied with a supply voltage comprised between 1V and 150 V.

12. The device according to claim 10, characterized in that, when said device is used for generating ultrasound in an aqueous or liquid medium, the capacitive micromachined ultrasonic transducer has:
a gap height of 100 nm;
an excitation voltage of 50 V;
a membrane width comprised between 13 and 35 μm;
a membrane thickness comprised between 200 and 800 nm; and
A Young's modulus of 200 GPa.

13. The device according to claim 10, characterized in that, when said device is used for generating ultrasound in an aqueous or liquid medium, the capacitive micromachined ultrasonic transducer has:
a gap height of 200 nm;
an excitation voltage of 100 V;
a membrane width comprised between 13 and 35 μm;
a membrane thickness comprised between 200 and 800 nm; and
A Young's modulus of 200 GPa.

14. The device according to claim 10, characterized in that, when said device is used for generating ultrasound in an aqueous or liquid medium, the capacitive micromachined ultrasonic transducer has:
a gap height of 300 nm;
an excitation voltage of 100 V;
a membrane width comprised between 20 and 30 μm;
a membrane thickness comprised between 300 and 550 nm; and
a Young's modulus of 200 GPa.

15. The device according to claim 10, characterized in that, when said device is used for generating ultrasound in a gaseous medium, the capacitive micromachined ultrasonic transducer has:
a gap height of 100 nm;
an excitation voltage of 50 V;
a membrane width comprised between 10 and 35 μm;
a membrane thickness comprised between 200 and 800 nm; and
a Young's modulus of 200 GPa.

16. The device according to claim 10, characterized in that, when said device is used for generating ultrasound in a gaseous medium, the capacitive micromachined ultrasonic transducer has:
a gap height of 200 nm;
an excitation voltage of 50 V;
a membrane width comprised between 20 and 40 μm;
a membrane thickness comprised between 300 and 600 nm; and
a Young's modulus of 200 GPa.

17. The device according to claim 10, characterized in that, when said device is used for generating ultrasound in a gaseous medium, the capacitive micromachined ultrasonic transducer has:
a gap height of 300 nm;
an excitation voltage of 100 V;
a membrane width comprised between 20 and 30 μm;
a membrane thickness comprised between 300 and 600 nm; and
a Young's modulus of 200 GPa.

18. The device according to claim 10, characterized in that it comprises:
a first supply module provided to supply the capacitive micromachined ultrasonic transducer with an excitation signal having a frequency lower than said resonance frequency;
a second supply module provided to supply the capacitive micromachined ultrasonic transducer with an excitation signal having a frequency centred around said resonance frequency; and
selection means for selecting one of said supply modules so that said capacitive micromachined ultrasonic transducer is supplied by only one of said supply modules at a time.

19. The device according to claim 10, characterized in that it comprises:
at least one first and at least one second capacitive micromachined ultrasonic transducer having an identical resonance frequency;
a first supply module provided to supply said at least one first capacitive micromachined ultrasonic transducer with an excitation signal having a frequency lower than said resonance frequency; and
a second supply module provided to supply said at least one second capacitive micromachined ultrasonic transducer with an excitation signal having a frequency centred around said resonance frequency.

20. A system for ultrasound medical imaging, comprising:
at least one device according to claim 10 for exciting a tissue or an organ of a human or animal subject; and
imaging means for taking images of said tissue or organ when said organ is excited.
